(19) United States
(12) Patent Application Publication
(10) Pub. No.: US 2022/0380718 A1
(43) Pub. Date: Dec. 1, 2022

(54) CONTINUOUS RECONSTITUTION OF PROCESS MATERIALS FROM SOLIDS

(71) Applicant: Universität für Bodenkultur Wien, Vienna (AT)

(72) Inventors: Daniel KOMUCZKI, Vienna (AT); Peter SATZER, Vienna (AT); Alois JUNGBAUER, Vienna (AT)

(21) Appl. No.: 17/787,549

(22) PCT Filed: Dec. 18, 2020
(86) PCT No.: PCT/EP2020/087141

§ 371 Date: Jun. 20, 2022

(30) Foreign Application Priority Data
Dec. 19, 2019 (EP) 19217901.8

(51) Int. Cl. C12M 1/00

(52) U.S. Cl. CPC C12M 99/00

(57) ABSTRACT

A system and a method for on-demand reconstituting solid process material is disclosed. The system comprises a feeding device for continuously feeding said solid process material, a mixing vessel, optionally a hold tank, optionally one or more mixing reactors, and optionally a sterile filter unit, wherein the system is configured to operate continuously.

### FIELD OF THE INVENTION

The present invention refers to a system and a method for continuously reconstituting process materials.

### BACKGROUND ART

Process materials such as cell culture media, buffers, substrates, stock solutions, nutrients, salts, polymers, chemicals or additives are of utmost importance in the chemical, biotechnological and food industry.

Such process materials are typically reconstituted from liquids, such as stock solutions. However, liquids require large storage space and/or tank sizes and have a limited shelf-life, which is for example due to limited stability of dissolved compounds or to light sensitivity. Therefore, liquids must be ordered regularly and are preferably stored under special conditions, such as refrigeration. In addition, the shipping of liquids is expensive and their handling is labor-intensive.

To reduce storage space and/or tank sizes, working from concentrated stock solutions is possible. However, stock solution concentrations are restricted by the solubility limit of the compounds to be dissolved.

To optimize supply chains, purchasing process materials in solid form is beneficial, since solids, such as powders or granulates, are less expensive, have a longer shelf life and require less storage space. However, the solid process materials need to be reconstituted to a liquid state and often need to be sterilized, e.g. for the application in biotechnology.

Reconstitution of solid process materials typically requires several steps, including adding a solvent, adjusting parameters such as concentration, pH value, additives, and finally sterilizing the reconstituted process materials. Sterilization of reconstituted process materials of particular importance in biotechnology and is e.g. performed by passing the liquid through a sterile filter.

Typically, solid process materials are reconstituted batch wise and are added to the respective reactor either in a batch wise manner or by inline mixing systems.

WO2017087040A1 discloses a mixing apparatus for reconstituting powdered cell culture media, which apparatus is operated in batch mode.

Current trends e.g. in the biopharmaceutical industry move towards continuous production of products, leading to increasing demands on the corresponding process materials. Large-scale batch wise reconstitution of process materials leads to high storage costs and labor costs, while a repeated batch wise reconstitution in smaller amounts may result in poor reproducibility due to e.g. human error or variations in the process material quality. In particular, batch wise process material reconstitution requires hold tanks for the reconstituted material before the material is transferred to a reactor. Hold tanks limit the process development in terms of cost, footprint and missing flexibility, especially during continuous processes.

Therefore, a method for continuous reconstitution of process materials is desirable. Such methods are still in its infancy. WO2019007786A1 describes a process for the continuous dissolution of a solid in a reaction medium, wherein the solid is provided in the form of a fixed bed, which process is intended to dissolve poorly soluble additives used in the chemical industry.

WO2013056469A1 discloses systems for preparing cell culture dish media and cell culture dishes in a batchwise-mode. U.S. Pat. No. 5,362,642A discloses a cell culture media containment system wherein powdered cell culture media and other constituents are introduced into the mixing bag, are mixed therein, and thereafter conveyed from the mixing bag into the storage bag undergoing sterilization. CN108893406A discloses a microbial ferment quantification production system and method.

However, there is still a need for a convenient and easy to handle system allowing continuous reconstitution of solid process materials, e.g., solid bioprocess materials.

### SUMMARY OF INVENTION

It is the objective of the present invention to provide a system and a method for continuously reconstituting a process material.

The objective is solved by the claimed subject-matter and as further disclosed herein.

The present invention provides a system for on-demand reconstituting solid process material, comprising
a. a feeding device for continuously feeding said solid process material;
b. a mixing vessel;
c. optionally a hold tank;
d. optionally one or more mixing reactors;
e. optionally a sterile filter unit; and
   wherein the system is configured to operate continuously.

The technical effect of the feeding device is to continuously add solid process material to the mixing vessel. The technical effect of the mixing vessel is to provide a liquid wherein the solid process material is added for reconstituting the process material. The technical effect of the hold tank is to provide a liquid which is added to the mixing vessel. The technical effect of the mixing reactors is to improve the reconstitution of the process material. The technical effect of the sterile filter unit is to sterilize the reconstituted process material.

In one embodiment described herein, the feeding rate can be directly regulated during the operation by adjusting the feeding device of step b).

In one embodiment described herein, the system is connected to a reactor. In one aspect, the reactor is a bioreactor, specifically a fermentation bioreactor operated in batch mode, fed-batch mode or continuous mode. In another aspect, the reactor is a hold tank for storing the reconstituted process material. In yet another aspect, the reactor is a reaction vessel, preferably a reaction vessel for downstream processing.

According to one aspect, the solid process material is an organic or inorganic material, or a combination thereof. According to a specific aspect, said solid process material is selected from the group consisting of a cell culture medium, a buffer, a nutrient, an additive, a substrate, a salt, a polymer, a chemical, and/or a bulk material, or any combination thereof. In another aspect, the solid process material is a cell culture medium, or a chemically defined cell culture medium, or a basal chemically defined cell culture medium. Specifically, said chemically defined cell culture medium may comprise any one or more of a carbohydrate, an amino acid, a vitamin, a fatty acid, an inorganic salt, a growth factor, a trace element, a protein, a peptide, a nucleic acid, a polymer and/or an organic salt.

In another embodiment described herein, the solid process material is a buffer, such as a buffer used in bioprocessing, or a buffer used in a chemical process. Specifically, said buffer may comprise any one or more of phosphate, sulphate, bicarbonate, acetate, lactate, citrate, malonic acid, formic acid, butanedioic acid, malonic acid, borate, tris, bis-tris, HEPES MES, MOPS, HEPPS BICINE, histidine, glutamate arginine, succinate, citrate, N-methyl piperazine, piperazine, imidazole, triethanolamine, diethanolamine, ethanolamine, 1,3-diamino-propane, piperidine, or any combination thereof, or any other suitable mineral acid or organic acid buffer.

According to one aspect of the invention, said solid process material is provided in the form of a powder, a slurry, a crystal, an organic polymer, an inorganic polymer, or a granulate. In one aspect, the solid process material is a powder or a granulate.

In one embodiment described herein, the feeding device is selected from the group comprising a screw conveyor, extruder, apron conveyor, pneumatic conveyor, roller conveyor, belt conveyor, pelletizer, compounder, gravimetric feeder, acoustic and ultrasonic vibration conveyor, rotary conveyor, electromagnetic conveyor, vertical conveyor. The solid process material is added to the mixing vessel by the feeding device by any mechanism such as gravimetric force, acoustic vibration, ultrasonic vibration, pulse inertia force, acoustic radiation force, electromagnetic force, vacuum force, weight, apron, belt, roller, rotary, vertical movement, or any combination thereof.

In another embodiment, a feeding hopper is connected to said feeding device.

In another embodiment, the feeding device is driven by a motor and the feeding rate is regulated by said motor, wherein said motor includes DC motors, AC motors and other motors such as a stepper motor, brushless motor, reluctance motor, universal motor.

In one embodiment of the system described herein, the feeding device is comprised in a confinement.

In another embodiment of the system described herein, said confinement is flushed by gas either with or without an overpressure.

According to one aspect, the system described herein comprises one or more tubular reactors as mixing reactors. Preferably the system comprises one or two tubular reactors which are connected to each other and are operated continuously. The technical effect of a tubular reactor is to provide a mixing device with a plug flow profile and without moving parts, which mixing reactor is stackable and therefore scalable, and allows a shortened reconstitution time and reduced process duration in comparison to non-tubular reactors.

In one embodiment, the system described herein comprises one or more integrated sensors for assessing one or more process parameters. The technical effect of said one or more integrated sensors is to allow in-line assessment of one or more process parameters. In one aspect, said one or more process parameters are selected from the group comprising temperature, pH, flow rate of a liquid, flow rate of a reconstituted process material, feeding rate of the feeding device, concentration of a reconstituted process material, spectroscopic properties of a reconstituted process material, conductivity of a liquid, conductivity of a reconstituted process material, redox potential, pressure, air moisture, and biomass.

Specifically, said integrated sensors are selected from the group consisting of a temperature sensor, a pH sensor, a flow rate sensor, a concentration sensor, a fluorescence sensor, an infrared light sensor, a sensor for inelastic scattering of monochromatic light, a conductivity sensor, a redox potential sensor, a pressure sensor, an air moisture sensor, and a biomass sensor, or any combination thereof. In one aspect, the system further comprises one or more units for controlling and adjusting process parameters, such as a temperature control unit, a pH control unit, a flow rate control unit, or a pressure control unit.

In one embodiment, the system described herein is a disposable and/or single-use system. The technical effect of a disposable and/or single-use system is the reduction of process time, labor and costs due to the avoidance of sterilization, cleaning and maintenance steps, the reduced risk of contamination, and the compatibility with disposable production systems, e.g. in the biopharmaceutical industry.

The present invention further provides a method for reconstituting a process material on-demand in a continuous mode, comprising the steps of
a. providing a system as described herein;
b. adding a solid process material in a continuous mode to the mixing vessel;
c. adding liquid in a continuous mode to the mixing vessel;
d. allowing said solid process material to dissolve in and/or to mix with said liquid in the mixing vessel to provide reconstituted process material; and
e. transferring the reconstituted process material in a continuous mode to the reactor.

In one aspect, said liquid is selected from the group comprising water, dissolved or partially dissolved buffer in a solvent, dissolved or partially dissolved chemically defined medium in a solvent, and/or recycled process stream, wherein said liquid is provided from a hold tank or from a reactor. According to a specific aspect, the liquid is water and is provided from a hold tank. According to another specific aspect, the liquid is a dissolved or partially dissolved buffer in water and is provided from a hold tank. According to yet another specific aspect, the liquid is a recycled process stream provided from a reactor, to which the system described herein is connected to. In one aspect, such reactor is a bioreactor, preferably a fermentation bioreactor. In another aspect, the reactor is a hold tank for storing the reconstituted process material. In yet another aspect the reactor is a reaction vessel, preferably a reaction vessel for downstream processing.

In one embodiment of the method described herein, the solid process material is an organic or inorganic material, or a combination thereof. According to a specific aspect, said solid process material is selected from the group comprising a cell culture medium, a buffer, a nutrient, an additive, a substrate, a salt, a polymer, a chemical, and/or a bulk material, or any combination thereof. In one aspect, the solid process material is a cell culture medium, or a chemically defined cell culture medium, or a basal chemically defined cell culture medium. Specifically, said chemically defined cell culture medium may comprise any one or more of a carbohydrate, an amino acid, a vitamin, a fatty acid, an inorganic salt, a growth factor, a trace element, a protein, a peptide, a nucleic acid, a polymer and/or an organic salt. In another preferred embodiment of the method described herein, the solid process material is a buffer, such as a buffer used in bioprocessing, or a buffer used in a chemical process. Specifically, said buffer may comprise any one or more of phosphate, sulphate, bicarbonate, acetate, lactate, citrate, malonic acid, formic acid, butanedioic acid, malonic acid, borate, tris, bis-tris, HEPES MES, MOPS, HEPPS BICINE, histidine, glutamate arginine, succinate, citrate, N-methyl piperazine, piperazine, imidazole, triethanolamine, diethanolamine, ethanolamine, 1,3-diamino-propane, piperidine, or any combination thereof, or any other suitable mineral acid or organic acid buffer.

According to one aspect of the method described herein, said solid process material is provided in the form of a powder, a slurry, a crystal, an organic polymer, an inorganic polymer, or a granulate. In one aspect, the solid process material is a powder or a granulate.

According to another aspect of the method described herein, the continuous operation mode is performed for at least 12 hours.

According to another method describes herein, the batch-wise production of the process media is avoided.

In one embodiment described herein, said reactor is a bioreactor comprising mammalian cells, bacterial cells, insect cells, fungal cells, algae or yeast; producing a product. Specifically, the product is a peptide, a protein, an oligonucleotide, a polynucleotide, a protein conjugate, a cell metabolite, a virus, a virus like particle, an exosome, a microorganism, a cell, or a tissue.

In a specific embodiment, said bioreactor comprises any of mammalian cells, bacterial cells, insect cells, fungal cells, algae or yeast, wherein said mammalian cells, bacterial cells, insect cells, algae or yeast produce a fermentation product selected from the group comprising a peptide, a protein, an oligonucleotide, a polynucleotide, a protein conjugate, and a cell metabolite. In one aspect, the fermentation product is a protein, e.g., an antibody, or a monoclonal antibody.

In another specific embodiment, said bioreactor comprises mammalian cells, bacterial cells, insect cells, fungal cells, algae or yeast, wherein the mammalian cells, bacterial cells, insect cells, algae or yeast are used as a product, e.g. for cell therapy.

In a specific aspect described herein, said process material is a chemically defined cell culture medium and said bioreactor is a fermentation bioreactor comprising mammalian cells producing a fermentation product. For example, said mammalian cells are CHO cells, and said fermentation product is a protein, e.g., an antibody, or a monoclonal antibody.

### BRIEF DESCRIPTION OF DRAWINGS

FIG. 1: A system for reconstituting a solid process showing a screw conveyer comprising a storage tank and/or a hopper as a solid feeding device for adding a solid process material to a mixing vessel, a hold tank or a WFI connected to the mixing vessel for supplying a liquid to the mixing vessel, a mixing vessel for reconstituting the solid process material in the liquid, a tubular reactor as a mixing reactor connected to the mixing vessel for improving the reconstitution of the process material, a filter unit for sterilizing the reconstituted process material, and a reactor, such as a bioreactor, a reaction vessel or a hold tank, to which the reconstituted process material is transferred.

FIG. 2: A calibration curve of the solid media feeding at 50, 150, 300 and 450 rpm.

FIG. 3: Profiles of pH (A) and osmolality (B) of a batch and continuously reconstituted basal medium. The arrow indicates the volume increase by pipetting and consequently the decrease in osmolality.

FIG. 4: Profiles of the viable cell density (VCD) (A), viability (B), the total amount of cells accumulated (cIVCD, C) and the average growth rate during the exponential phase (D). Error bars indicate biological quadruplets (n=4) for batch and duplicates (n=2) for continuous cultures.

FIG. 5: Concentration profiles of Glc (A), osmolality (B) and final concentrations of Lac (C) and antibody concentration (D). Error bars indicate biological quadruplets (n=4) for batch and duplicates (n=2) for continuous cultures.

FIG. 6: Amino acid (AA) profiles of non-essential AA (A) and essential AA (B) after reconstitution continuously or batch wise. Error bars indicate biological quadruples (n=4) for batch and duplicates (n=2) for continuous cultures.

FIG. 7: Concentration profiles of non-essential (A) and essential (B) amino acids at the end of the cultivation at day 7. Error bars indicate biological quadruplets (n=4) for batch and duplicates (n=2) for continuous cultures.

FIG. 8: Monitoring of pH (▴), conductivity (▪) and UV absorbance (280 nm, ●) of the continuous on-demand reconstitution of a chemically defined media over a duration of 12 h. Dashed boxes indicate the temporarily failure of the system by arching and increase in relative humidity within the confinement. Due to visualization purposes the number of data points has been decreased (n=29108 data points).

FIG. 9: Calibration curve and linear regression of the solid media long-term feeding. For illustration purposes the number of data points has been reduced (n=727).

FIG. 10: Relative abundance of non-essential (A) and essential (B) individual amino acid of long-term continuously on-demand and batchwise reconstituted medium. Concentration of the individual amino acid profiles after long-term reconstitution and the diluted batch medium (C, D).

FIG. 11: The profile of the (A) Viable cell density, (B) viability, (C) glucose and (D) lactate profiles (n=2).

FIG. 12: AA profiles of non-essential AA (A) and essential AA (B) at the day of harvest of continuously on-demand or batch wise cultured CHO-K1 cells.

FIG. 13: Sheathless CE-MS of intact mAbs. Deconvoluted mass spectra of (A) continuous on-demand reconstituted and (B) batchwise reconstituted samples derived from controlled conditions after sheathless CE-MS separation. The inset shows the base peak electropherograms of both samples. (C) Relative intensity of the glycoforms of mAbs produced with continuous on-demand reconstituted medium (black) or batchwise reconstituted medium (white).

FIG. 14: Reconstitution of five different buffering agents at various motor speeds (A) and reconstitution of sodium chloride at five different motor speeds.

FIG. 15: Salt gradient generated by in-situ preparation directly from solid buffer components for the separation of two proteins.

FIG. 16: Salt elution gradients of different length performed by the device (black) and the conventional chromatographic workstation ÄKTA (dashed) for the separation of two proteins at 1 mL (A, C, D) and 10 mL CV (B).

FIG. 17: Step elution gradients performed by the device (black) and the conventional chromatographic workstation ÄKTA (dashed) with imidazole for the chromatographic purification of a protein (A) and elution profile of imidazole (B).

### DESCRIPTION OF EMBODIMENTS

The present invention relates to a system and a method for continuously reconstituting a process material.

Unless indicated or defined otherwise, all terms used herein have their usual meaning in the art, which will be clear to the skilled person.

The terms “reconstitute” and “reconstitution” as used herein refer to the process through which a solid material is converted to a liquid form through the addition of, and mixing with, a liquid. The term “reconstituted” describes a material produced by reconstituting. The term “solid” as used herein refers to a dehydrated material. The term “liquid” is herein used to refer to any fluid material capable of dissolving, dispensing, suspending, colloidally suspending, emulsifying or otherwise blending the solid material. For example, a liquid may include water, dissolved or partially dissolved buffer in a solvent such as water, dissolved or partially dissolved chemically defined medium in a solvent such as water, and/or a recycled process stream.

Once reconstituted, the solid material may be one or more of dissolved, dispersed, suspended, colloidally suspended, emulsified, or otherwise blended within the matrix of the liquid. Therefore, the resulting reconstituted material may be characterized as a solution, a dispersion, a suspension, a colloidal suspension, an emulsion, or a homogeneous blend, or any combination thereof.

As used herein, the term “continuous” or “continuously” refers to a process run on a continuous flow basis, i.e. a process which is not taking place within any defined period of time, in contrast to batch, intermittent, or sequenced processes. The product of a continuous process is generally also removed continuously from the process. Batch processes are in contrast to continuous processes typically carried out for a specified period of time after which the product is removed from the process. In embodiments of the present invention, the terms “continuous”, “continuously”, and the like, can mean a mode of reconstituting a solid process material in a mixing vessel in such a manner so as to continuously produce a reconstituted process material in a system described herein and continuously transfer said reconstituted process material out of the mixing vessel.

The term “process material” is herein understood as a material which is used in a chemical and/or a bioprocess. For example, the process material may include an organic and/or inorganic material, or any combination thereof. The term “organic material” shall refer to materials comprising or consisting of organic carbon molecules. Non-limiting examples of organic materials include alcohols, ketones, aldehydes, fatty acids, esters, carboxylic acids, ethers, carbohydrates, amino acids, peptides, proteins, lipids, monosaccharides, oligosaccharides, polysaccharides, nucleic acids, organic salts, and organic polymers such as thermoplasts, elastomers, or resins. The term “inorganic material” generally refers to materials that are not organic compounds or organic materials. Non-limiting examples of inorganic materials include minerals, salts, metals, and inorganic polymers.

Specifically, a process material is selected from the group comprising a cell culture medium, a buffer, a nutrient, an additive, a substrate, a salt, a polymer, a chemical, and/or a bulk material, or any combination thereof. In a preferred embodiment, the process material is a cell culture medium, even more preferably a chemically defined cell culture medium. The term “cell culture medium” is herein understood as a medium for culturing cells containing a carbon/energy source, and nutrients that maintain cell viability, support proliferation, growth and/or the production of a fermentation product e.g. by biotransformation of a carbon source. The term “cell culture medium” as used herein also includes a concentrated cell culture medium (“feed medium”). The term “chemically defined cell culture medium” shall refer to a cell culture medium consisting of ingredients free of animal origin. A chemically defined cell culture medium may contain any of the following in an appropriate combination: a carbohydrate, such as e.g. glucose, lactose, sucrose, and fructose, an amino acid, a vitamin, a fatty acid, an inorganic salt, a growth factor, a trace element, a protein, a peptide, a nucleic acid, a polymer, and/or an organic salt.

In another embodiment, the process material is a buffer, herein understood as a solution which resists changes in pH by the action of its conjugate acid-base range. Such buffers comprise salts of strong or weak acids or bases, charged amino acids or amines, and/or Good's buffers, herein understood as buffers for biochemical and biological applications. Some of the buffer components may be solids at room temperature. Salts containing sodium, ammonium, and potassium cations are often used in preparing a buffer. Non-limiting examples of suitable buffers include phosphate, sulphate, bicarbonate, acetate, lactate, citrate, malonic acid, formic acid, butanedioic acid, malonic acid, borate, tris, bis-tris, HEPES MES, MOPS, HEPPS BICINE, histidine, glutamate arginine, succinate, citrate, N-methyl piperazine, piperazine, imidazole, triethanolamine, diethanolamine, ethanolamine, 1,3-diamino-propane, piperidine, or any combination thereof, or any other suitable mineral acid or organic acid buffer. Said buffers are used to control pH values.

In yet another embodiment, the process material is an additive, herein understood as a substance or a mixture of substances added to a biotechnological or chemical process in relatively small amounts, e.g. to impart or improve desirable properties or suppress undesirable properties, or to generate a phase transition. Additives may be used as precipitants for protein precipitation and crystallization or flocculation. Non-limiting examples of additives include polyethylene glycol (PEG), bivalent ions, zinc, calcium, ammonium sulphate, potassium sulphate, sugars and other polyols.

In one aspect described herein, the solid process material is provided in the form of a powder, a slurry, a crystal, an organic polymer, an inorganic polymer, or a granulate. Preferably, the solid process material is a powder or a granulate. A powder is herein understood as a flowable material, preferably with a density in the range of 0.02 g/mL to 3 g/mL, more preferably in the range of 0.1 g/mL to 0.7 g/m L.

The system for continuously reconstituting a process material as described herein comprises a solid process material, a feeding device, a mixing vessel, optionally a hold tank, optionally one or more mixing reactors and optionally a sterile filter unit. The system according to the present invention is configured to operate continuously.

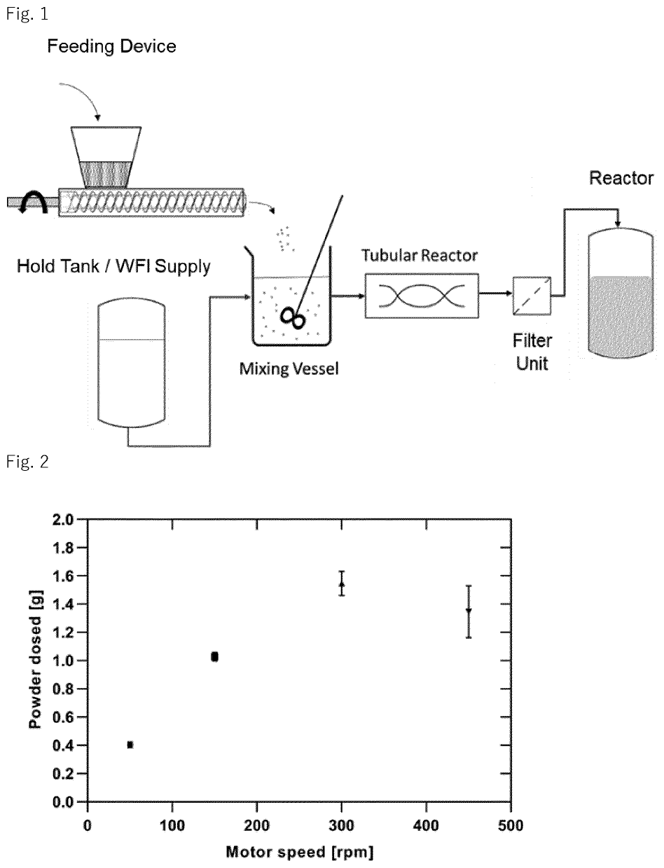

The system described herein comprises at least one feeding device. Said feeding device is positioned in the system in such a way as to transfer solid process material to the mixing vessel. The feeding device may e.g. be positioned above the mixing vessel and may, or may not, be in direct (physical) contact with mixing vessel. Non-limiting examples of a suitable feeding device include a dispenser, screw conveyer, extruder, apron conveyor, pneumatic conveyor, roller conveyor, belt conveyor, pelletizer, compounder, gravimetric feeder, acoustic and ultrasonic vibration conveyor, rotary conveyor, electromagnetic conveyor, vertical conveyor. The feeding device allows for the continuous movement of solid process material by any mechanism such as gravimetric force, acoustic vibration, ultrasonic vibration, pulse inertia force, acoustic radiation force, electromagnetic force, vacuum force, weight, apron, belt, roller, rotary, vertical movement, or any combination thereof. In one aspect, the feeding device is a screw conveyer. In a specific embodiment described herein, as displayed in FIG. 1, the feeding device is a screw conveyer positioned above, but not directly connected to, the mixing vessel, and the material is transferred to the mixing vessel by gravity.

The mixing vessel allows reconstituting the solid process material in a liquid and may contain one or more mixing elements selected from the group consisting of a mechanical stirrer, an electromagnetic stirrer, a submersible stirrer, and a biological stirrer. The volume of a suitable mixing vessel may be in the range of 0.04 L to 8000 L. Non-limiting examples include a volume of 0.04 L, 0.1 L, 0.5 L, 1 L, 5 L, 10 L, 50 L, 70 L, 100 L, 200 L, 300 L, 400 L, 500 L, 1000 L, 1500 L, 2000 L, 3000 L, 4000 L, 5000 L, 6000 L, 7000 L, or 8000 L.

Specifically, the system described herein comprises one or more pumps for continuously transferring the reconstituted process material out of the mixing vessel. Non-limiting examples for a suitable pump include a peristaltic pump, a piston pump, a vacuum pump, a screw pump, a gear pump, and an eccentric screw pump.

Specifically, the liquid in which the process material is reconstituted is transferred continuously to the mixing vessel. Specifically, the liquid may be transferred at a flow rate in the range of 0.1 to 4 mixing vessel volume exchanges per day, or at a flow rate of up to 50 mixing vessel volume exchanges per day. The liquid may be supplied from a hold tank or from a reactor to which the system described herein is connected to. If the liquid is supplied from a reactor, said liquid is preferably a recycled process stream.

In one embodiment, the system described herein comprises a hold tank, which hold tank contains a liquid in which the process material is to be reconstituted. For example, said liquid may include water, dissolved or partially dissolved buffer in a solvent such as water, or dissolved or partially dissolved chemically defined medium in a solvent such as water. Said hold tank is connected to the mixing vessel, e.g. by one or more suitable tubes. The connection of the hold tank with the mixing vessel by one or more suitable tubes will be clear to the skilled person. In one aspect, the volume of said hold tank is in the range of 0.1 L to 1000 L. Non-limiting examples include a volume of 0.1 L, 0.5 L, 1 L, 2 L, 5 L, 10 L, 50 L, 100 L, 150 L, 200 L, 300 L, 400 L, 500 L, 600 L, 700 L, 800 L, 900 L, or 1000 L. Specifically, the system may additionally comprise one or more pumps for continuously transferring said liquid to the mixing vessel.

The system described herein optionally comprises one or more mixing reactors which are operated continuously, wherein a first mixing reactor is connected to the mixing vessel and any further mixing reactors are connected to each other and positioned in a row. The connection between the mixing vessel with the first mixing reactor and/or the connection between the mixing reactors is e.g. provided by one or more suitable tubes. The reconstituted process material may be transferred from the mixing vessel to the first mixing reactor and to any further mixing reactors by one or more pumps, preferably at a flow rate in the range of 0.1 to 4 vessel exchanges per day. Said one or more mixing reactors may be a tubular reactor, a continuous stirred tank reactor, or a combination thereof. Said one or more mixing reactors allow for a complete reconstitution of the process material, e.g. in case of reconstituting a poorly soluble process material. The volume of such mixing reactor is typically in the range of 0.04 L to 200 L. Non-limiting examples include a volume of 0.04 L, 0.1 L, 0.5 L, 1 L, 5 L, 10 L, 20 L, 30 L, 40 L, 50 L, 75 L, 100 L, 125 L, 150 L, 175 L, or 200 L.

With reference to FIG. 1, the system according to a specific embodiment described herein contains a tubular reactor as a mixing reactor. The advantage of a tubular reactor is a plug flow profile and the avoidance of moving parts, which reduces the risk of technical complications. The tubular reactor allows for a shortened reconstitution time and reduced process duration, and superior mixing. In a specific embodiment described herein, the tubular reactor is stackable and therefore scalable based on user requirements, allowing a reduced operational footprint compared to conventional reconstitution processes.

The system according to the present invention may be manufactured by any method comprising, but not limited to, 3D printing, additive manufacturing, subtractive manufacturing, injection molding, or any combination thereof. Each part of the system described herein may comprise or consist of any material including, but not limited to, a metal, an alloy such as stainless steel, a plastic, a glass, a ceramic, or any combination thereof. Preferably, the system as described herein comprises stainless steel.

In another embodiment, the system described herein is a disposable and/or single use system, consisting of disposable materials such as polytetrafluoroethylene (PTFE), polyvinylidene fluoride (PVDF), polypropylene (PP), polyethylene (PE), silicone, or ethylene vinyl acetate copolymers (EVA), or any combination thereof, or any other disposable material. Advantages of a disposable system include the avoidance of sterilization, cleaning and maintenance steps, leading to reduced process times due to increase in productivity, and to reduced labor time, costs and materials, e.g. by avoiding large amounts of water for cleaning. Disposable systems further require less space, reduce the risk of contamination, and are compatible with disposable production systems, e.g. in the biopharmaceutical industry.

The system described herein optionally comprises a sterile filter unit for sterilizing the reconstituted process material. Said filter unit may be provided as a membrane or a filter, comprising one or more materials selected from the group of polytetrafluoroethylene (PTFE), polyvinylidene fluoride (PVDF), polypropylene (PP), poly(ether-sulfone)(PES), or any other suitable material. Specifically, said filter unit may be positioned after the mixing vessel, and connected to the mixing vessel, e.g. by a tube. In another embodiment, said sterile filter unit may be positioned after one or more mixing reactors, and connected to a mixing reactor (e.g. by a tube), e.g. as displayed in the specific example in FIG. 1.

In one aspect, the system according to the invention comprises one or more integrated sensors for assessing process parameters. The term “integrated” sensor refers to as a sensor which is integrated within or part of the system described herein. Specifically, an integrated sensor may be positioned inside the mixing vessel, inside the hold tank, and/or before, after, beneath or inside the optional mixing reactor, and/or before or after the optional filter unit. An integrated sensor allows for a convenient in-line assessment of process parameters. The term “in-line” as used herein refers to the possibility of continuously measuring a process parameter without drawing a sample, in contrast to off-line analysis, which includes drawing a sample for external analysis. Non-limiting examples of integrated sensors include a temperature sensor, a pH sensor, a flow rate sensor, a concentration sensor, a fluorescence sensor, an infrared light sensor, a sensor for inelastic scattering of monochromatic light (e.g. Raman probe), a conductivity sensor, a redox potential sensor, a pressure sensor, an air moisture sensor, and a biomass sensor. Integrated sensors allow assessing process parameters such as temperature, pH, flow rate of a liquid, flow rate of a reconstituted process material, feeding rate of the feeding device, concentration of a reconstituted process material, spectroscopic properties of a reconstituted process material, conductivity of a liquid, conductivity of a reconstituted process material, redox potential, pressure, air moisture, and biomass.

The term “assessment” or “assessing” as used herein refers to measuring, analyzing and reacting to a determined process parameter. In one aspect, the system further comprises one or more units for controlling and adjusting process parameters, such as a temperature control unit, a pH control unit, a flow rate control unit, or a pressure control unit. Said one or more units for controlling and adjusting process parameters may be connected to said integrated sensors.

In one embodiment, the system as described herein is connected to a reactor, or optionally more than one reactor, used in an industrial biotechnological or chemical process, which process may be a batch, fed-batch or continuous process. In one aspect, there is a further connection from said reactor to the mixing vessel of the system described herein for continuously transferring a recycled process stream from the reactor to the mixing vessel, allowing the reconstitution of a process material in said recycled process stream.

In one embodiment, said reactor is a bioreactor for producing a product such as a peptide, a protein, an oligonucleotide, a polynucleotide, a protein conjugate, a cell metabolite, a virus, a virus like particle, an exosome, a microorganism, a cell, or a tissue. Specifically said bioreactor has a typical volume ranging from 1 L to 50,000 L. Non-limiting examples include a volume of 1 L, 2 L, 3 L, 4 L, 5 L, 6 L, 7 L, 8 L, 9 L, 10 L, 15 L, 20 L, 25 L, 30 L, 40 L, 50 L, 60 L, 70 L, 80 L, 90 L, 100 L, 150 L, 200 L, 250 L, 300 L, 350 L, 400 L, 450 L, 500 L, 550 L, 600 L, 650 L, 700 L, 750 L, 800 L, 850 L, 900 L, 950 L, 1000 L, 25 1500 L, 2000 L, 2500 L, 3000 L, 3500 L, 4000 L, 4500 L, 5000 L, 6000 L, 7000 L, 8000 L, 9000 L, 10,000 L, 15,000 L, 20,000 L, and/or 50,000 L.

According to one aspect, the bioreactor is a fermentation bioreactor, also referred to as “fermenter” or “fermentation unit”, for producing a fermentation product, which fermentation bioreactor is operated in batch, fed-batch or continuous mode. The term “batch mode” in the context of a fermentation process is herein to be understood as a cell culture process by which a small amount of a cell culture solution is added to a medium and cells are grown without adding an additional medium or discharging a culture solution during culture. “Fed-batch mode” in the context of a fermentation process refers to a culture technique starting with cell growth in the batch phase, followed by a “fed” phase during which the cell culture is in continuous mode, wherein the cell culture medium is continuously added (“fed”) to the bioreactor. “Continuous mode” in the context of a fermentation process is a cell culture process by which a medium is continuously added and discharged during culture. Examples for continuous mode processes include perfusion and chemostat processes.

According to one aspect described herein, said fermentation bioreactor comprises mammalian cells, bacterial cells, insect cells, fungal cells, algae or yeast producing a fermentation product. Non-limiting examples of a fermentation product may include a peptide, a protein, an oligonucleotide, a polynucleotide, a protein conjugate, and a cell metabolite. In one aspect, the fermentation product is a protein, e.g., an antibody, or a monoclonal antibody.

In a specific embodiment, the system and method described herein is used for continuously reconstituting a cell culture medium or a buffer which is continuously transferred to a fermentation bioreactor comprising cells producing a fermentation product. In a specific example as described in the Examples section, said cell culture medium is a basal chemically defined cell culture medium and said bioreactor is a fed-batch bioreactor comprising CHO cells producing a monoclonal antibody, such as immunoglobulin G1. The term “basal” refers to a chemically defined cell culture medium that is designed to support growth but is not enriched in e.g., amino acids.

According to another aspect, said bioreactor is a bioreactor comprising mammalian cells, bacterial cells, insect cells, fungal cells, algae or yeast, wherein the mammalian cells, bacterial cells, insect cells, algae or yeast are used as a product, e.g. for cell therapy. Specifically, said bioreactor is a bioreactor for the production of biomass such as cells, microorganisms, viruses, a virus like particles, exosomes, or tissue. In a specific embodiment, the system and method described herein are used for continuously reconstituting a cell culture medium or a buffer, which cell culture medium or buffer is continuously transferred to a bioreactor for biomass production.

In another embodiment described herein, said reactor is a hold tank for storing a reconstituted process material. Said hold tank may have a volume in the range of 0.02 L to 1000 L. Non-limiting examples include a volume of 0.02 L, 0.05 L, 0.1 L, 0.5 L, 1 L, 5 L, 10 L, 20 L, 50 L, 70 L, 100 L, 150 L, 200 L, 300 L, 400 L, 500 L, 600 L, 700 L, 800 L, 900 L or 1000 L.

In a specific embodiment, the system and method described herein are used for continuously reconstituting a cell culture medium, a buffer, or a stock solution, which cell culture medium or buffer is continuously transferred to a hold tank.

In yet another embodiment said reactor is a reaction vessel for downstream processing. The term “downstream processing” as used herein relates to process steps carried out after producing a product in a reactor in order to purify or modify said product. A non-limiting example for downstream processing is continuous chromatography or continuous ultrafiltration or dialfiltration, continuous precipitation, flocculation, crystallization, or virus inactivation. In a specific embodiment, the system and method described herein are used for continuously reconstituting a buffer, which buffer is continuously transferred to a reaction vessel for downstream processing. Specifically, the reaction vessel for downstream processing is a packed bed (column chromatography) or a stirred tank reactor, e.g. combined with a filter dialfiltration.

In a further embodiment, said reactor is a reaction vessel for food production. In a specific embodiment, the system and method described herein are used for continuously reconstituting a nutrient or an additive, which nutrient or additive is continuously transferred to a reaction vessel for food production.

The present invention provides a method for continuously reconstituting a process material, comprising the steps of providing a system as described herein, continuously adding a solid process material to the mixing vessel as described herein, continuously adding liquid to the mixing vessel as described herein, continuously allowing said solid process material to mix with said liquid in the mixing vessel to provide reconstituted process material as described herein, and optionally continuously transferring the reconstituted process material into a reactor as described herein.

In one aspect, the method described herein further comprises assessing and controlling one or more process parameters using one or more integrated sensors and/or control units as described herein.

Specifically, the system and method described herein allow changing the process material composition during continuous reconstitution by changing one or more parameters such as the feeding rate of a feeding device, the flow rate of a liquid, the feeding rate of another process material, the mixing speed, temperature, and/or the volume of liquid in the mixing vessel.

In another aspect, a hopper is connected to the feeding device of the invention presented herein. Such a hopper may be added to the feeding device provided herein to store and to add the solid process material for the on-demand reconstitution.

In another aspect, the feeding device is driven by a motor and the feeding rate can be regulated by said motor. Non-limiting examples of said motor may include DC motors, AC motors and other motors such as a stepper motor, brushless motor, reluctance motor, universal motor.

In another aspect, the term “feeding rate” as used herein is defined as the amount of process material added to the mixing vessel per time unit and is described by the units g min⁻¹ if not stated otherwise.

In another aspect, the feeding device is comprised in a confinement. The confinement may be flushed by gas either with or without overpressure. Such a confinement may be of any material and shape known in the art for providing a confinement for a device that can be flushed by a gas. Non-limiting examples of such a confinement may be a cubic or cuboidal plastic box or a confinement of any other material and shape.

One embodiment of the invention relates to a method as described herein, wherein the continuous operation mode is performed for at least 12 hours. It is further envisaged that the continuous operation method of the present invention may be performed for at least 2 days, 3 days, 4 days, 5 days, 6 days, 1 week, 2 weeks, or for at least one months.

A further advantage of the inventive method is the avoidance of a batch-wise production of the process media which leads to reduced capital costs and a footprint reduction of the individual unit operations.

According to specific examples described in the Examples section, a system consisting of a solid feeding device, a mixing vessel, a hold tank and a tubular reactor for continuous media or buffer preparation directly from solids is disclosed. The advantages of this set up are that a gradual change of components of the reconstituted material in e.g. continuous processing can be achieved by changing the media composition and therefore limitations in terms of solubility, availability but also stability can be prevented. Moreover, the reconstitution of the material is facilitated directly from solids, thus, also environmental influences can be minimized by reducing the foot print of the buffer preparation. Even more, by using process analytical technology (PAT) and quality by design (QbD) a reconstitution of process materials can be monitored and controlled in-line. Another advantage of continuous media reconstitution is an improved and optimized process, potentially resulting in superior product quality. According to a specific embodiment described in the Examples section, a basal chemically defined cell culture medium was reconstituted both batch wise and continuously, and the process performance was compared for two CHO cell lines producing two different monoclonal antibodies immunoglobulin G1. Chinese hamster ovary (CHO) cell lines were chosen since CHO cells are applied for the production of therapeutic species (TP-S) in the biopharmaceutical industry, such as monoclonal antibodies.

### EXAMPLES

The Examples which follow are set forth to aid in the understanding of the invention but are not intended to, and should not be construed to limit the scope of the invention in any way. The Examples do not include detailed descriptions of conventional methods. Such methods are well known to those of ordinary skill in the art.

### Example 1—Materials and Methods for Reconstituting a Chemically Defined Cell Culture Medium

### Cell Lines and Media

A CHO-K1 and a CHO-S cell line (Antibody Lab GmbH, Austria) expressing different monoclonal antibodies (mAb) immunoglobulin G1 (IgG1) were used. For pre-cultures and N-stage the commercially available medium Dynamis™ AGT™ (Gibco®) supplemented with 4 mM L-Glutamine (Gln) and 1% Anti-Clumping Reagent was used.

### Pre-Cultures

After thawing, cells were washed with freshly prepared basal medium at 180 g (5 min, 23° C.). The supernatant was discarded and cells were resuspended in fresh basal medium supplemented with 1% G148 and 8 mM Gln. All cell lines were grown in 125 mL, 250 mL and 500 mL Erlenmeyer shake flasks (Corning, United States of America). Cell cultures were grown at 34.5° C., 5% CO₂, 70% humidity at 200 rpm in a MaxQ 2000 CO2 (Thermo Fisher, United States of America) 19 mm orbital shaker and passaged in a 3-day rhythm for 4 consecutive passages.

### Fed Batch Experiment

At the day of inoculation cells were centrifuged at 180 g (5 min, 23° C.). To ensure same starting conditions, cells were suspended and concentrated in the spent medium. The same volume was added for each inoculation in a 1:10 split ratio, respectively. Additionally, Gln and Anti-Clumping Reagent were spiked at 4 mM and 1%, respectively, before media equilibration. Fed-batch experiments were carried out using Spin Tubes (TubeSpin® Bioreactor 50, TPP) over a duration of 7 days. Working volume (wv) was set at 15 mL and all experiments were carried out in biological quadruplets. Media were equilibrated for 2 h at 36.5° C. For fed batch experiments cells were inoculated at a seeding cell density (SCD) of 7×10⁵ cells mL⁻¹. Glucose levels were maintained above 3 g L⁻¹ by bolus addition of a concentrated glucose stock solution (400 g L⁻¹), if glucose concentration was determined <3 g L⁻¹. Temperature was kept at 36.5° C. and the orbital shaker speed was set to 300 rpm. Notably, to prevent disguising effects no concentrated feed medium was introduced.

TABLE 1
Experimental set-up of the fed-batch cultivation in spin
tubes. Each set-up was performed in quadruplets using either
the batch or continuously reconstituted basal medium.

| | Cell line/ | | | |
|---|---|---|---|---|
| | Protein of | SCD | Basal | Basal |
| Set up | Interest | (×10⁵ | Medium | Medium |
| (n = 4) | (POI) | cells · mL⁻¹) | (Batch) | (Continuous) |
| 1 | CHO-K1 | 7 | — | X |
| 2 | Adalimumab | | X | — |
| 3 | | | | |
| 4 | | | | |
| 5 | | | | |
| 6 | | | | |
| 7 | CHO-S | | | |
| 8 | Trastuzumab | 7 | — | X |
| 9 | | | X | — |
| 10 | | | | |
| 11 | | | | |
| 12 | | | | |

### Estimation of Parameters

The growth rate during the exponential phase was defined as Eq. (1):

d dtCxv=μ EXP Cxv

wherein Cxv is the concentrations of viable cells in the bioreactor and μEXP is the average cell specific growth rate (d⁻¹) during the exponential phase. Rearranging Eq. (1), integrating and applying the natural logarithm results in μEXP between two timepoints (tᵢ₊₁−tᵢ) Eq. (2):

$$\frac{LN {( {C}_{xti + 1} )}_{Cxt}}{μ EXP = ( {t}_{i} + 1 - {t}_{ii} )}$$

The average cell specific production/consumption rates of (q_{MX}; pg cell⁻¹ day⁻¹) were calculated by plotting the cumulative integral of the viable cell density (cIVCD) against the total amount of production/consumption using linear regression. Negative values mean consumption and positive values mean production.

### In-Process Analytics

Samples were drawn daily for analysis of cell viability, glucose and metabolite concentrations. Product concentration was determined at end of the cultivation at day 7 (Spin Tubes) and day 9 (Bioreactors), respectively. Cell concentrations and viabilities were measured during pre-cultures and batch experiments using a Vi152 Cell™ (Beckman-Coulter) using the trypan blue exclusion method. Daily glucose levels were analyzed by the blood glucose monitoring system Contour X (Bayer). Osmolality was measured using the Single154 Sample Micro-Osmometer OsmoTECH® (Advanced Instruments, United States of America) and antibody concentrations were measured via a Protein A affinity HPLC (Thermo Fischer, United States of America). Cystein was analyzed in form of its dimers ((Cys)₂) due to the instability of Cys. Ammonium hydroxide levels were measured by a Cedex Bio Analyzer (Roche, Basel, Switzerland). Samples were sent to a laboratory for carbohydrate and amino acid quantification.

### Experimental Setup of Feeding Device for Reconstitution of Chemically Defined Cell Culture Media

FIG. 1 shows the basic system for reconstituting a solid process material as used in the examples regarding the reconstitution of culture media. The system used in the examples comprises a screw conveyer as a feeding device, a hold tank or a supply of water for injection (WFI), a mixing vessel, a tubular reactor, a filter unit, and as operation unit a bioreactor, spin columns or a continuously stirred tank reactor (CSTR) to which the reconstituted process material is transferred. Prior to reconstitution, several calibrations of the screw conveyer at various rpm were performed. Thereby, solids from a commercially available basal cell culture medium (Dynamis AGT, Thermo Fisher, United States of America) was put into the hopper of the feeding device and calibration experiments were conducted. The solids were fed for 2 min at 50, 150, 300 and 450 rpm in 5 technical replicate runs and the solid dosed were weight afterwards.

The calibration experiments were performed using an Entris® Precision balance (Sartorius, Gottingen, Germany) connected to a Raspberry Pi 3 for data acquisition. As can be seen from FIG. 2 with increase rpms the accuracy of the dispensing decreased. Osmolality was confirmed using an OsmoTech® single sample osmometer (Advanced Instruments, Norwood, United States). The data was collected on-line using the Simple Data Logger software (Smartlux SARL, Born, Luxembourg). For the continuous on-demand reconstitution, the solids were fed into a miniaturized continuously stirred tank reactor (CSTR) with a magnetic stirrer and bottom outlet. The media was reconstituted according to the manufacturer's recommendation and a residence time in the continuous reconstitution according to the recommended mixing time.

### Example 2—Comparison of Batch and Continuous Cell Culture Medium Reconstitution

The batch medium was reconstituted according to the manual provided by the vendor. For the continuously reconstituted medium a combination of a mixing vessel, screw conveyor and tubular reactor was used. Thereby, one peristaltic pump resupplied the mixing vessel with fresh water for injection (WFI) and a second pump transported the medium through the tubular reactor as illustrated in FIG. 1.

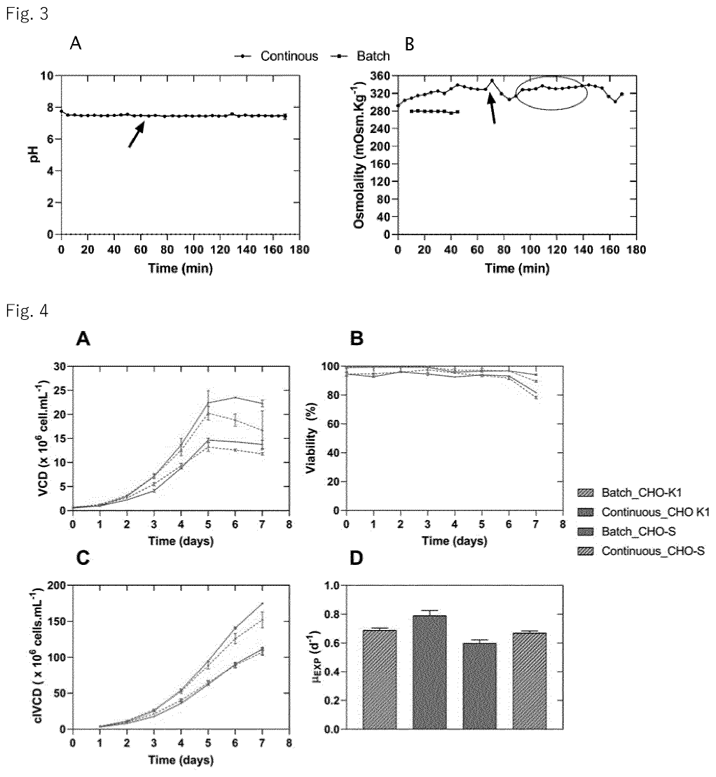

Mixing in the mixing vessel was achieved by a magnetic stirrer set at 400 rpm and the volume (VLM) was set constant at 40.7 mL by manual adjustment of the feed pump. The flow rates were set for both pumps at 8.7 mL min⁻¹ allowing a mixing time in the mixing vessel of 5 min and in the tubular reactors for 30 min which equals the minimum mixing time recommended by the vendor for batch reconstitution of the medium. The screw conveyer was geared with a stepper motor using a cog wheel and controlled by the single-board computer Raspberry Pi 3 with a python script. Medium powder was conveyed from the top by a screw conveyer at an average feeding rate of 0.202 g min⁻¹ (±0.02) (FIG. 1). The total runtime tested in this proof of concept for continuous reconstitution was 2.9 h. After a ramp-up phase of 40 min continuously reconstituted medium was collected in Falcon tubes in 40 mL aliquots. The samples were analyzed for pH and osmolality (see In-Process Analytics) and compared to a medium reconstituted in batch-mode under identical conditions. It is important to state that after the ramp-up phase a decrease in V_{LM} was observed (FIG. 3). Consequently, the feed rate was manually adjusted to ensure a constant volume. Additionally, the volume was increased by pipetting after 66 min which resulted in a decreased VLM over whole duration of the process. Nevertheless, after a slight fluctuation between minute 66 and 88 a relative constant volume was achieved. Therefore, the aliquots (indicated in FIG. 3 with a circle) during the constant osmolality and pH profiles were pooled and sterile filtered using a 0.8 μm Ministart® Syringe Filter (Satorius, Germany) and stored at 7° C.

### Example 3—Use of Reconstituted Cell Culture Medium in Fed-Batch Experiments

The aim of this experiment was to evaluate and to compare the batch wise and continuously reconstituted chemically defined cell culture media of Examples 1-2 in terms of their performance in fed-batch fermentations with two different CHO cell lines (CHO-K1 and CHO-S) producing antibodies.

### Fed-Batch Experiments

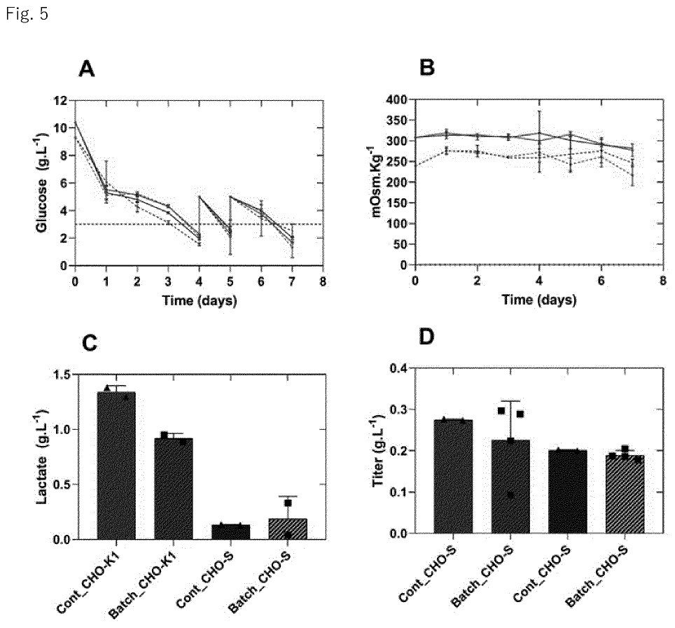

It can be clearly seen from FIG. 4 that irrespective of the reconstitution mode of the chemically defined medium (CDM), both cell lines lead to a comparable growth profile during the fed-batch experiments. Important to state is that two biological replicates in both cell lines using the continuously reconstituted medium were contaminated after 4 and 6 days which were, as a result, terminated. Nevertheless, the remaining cultures (duplicates) lasted for the whole duration of the process and lead to a slightly superior culture performance in comparison to cultures using a batch wise reconstituted medium. For the CHO-K1 cell line this resulted in an average maximum viable cell density (MVCD) of 23.50 (±0.06)×10⁶ cells mL⁻¹ for the continuous and 20.25 (±1.22)×10⁶ cells mL⁻¹ for the batch medium. Whereas, the CHO-S cell cultures of resulted in 12.59 (±0.69)×10⁶ cells mL⁻¹ and 14.25 (±0.275)×10⁶ cells mL⁻¹. Besides that, for both CHO-K1 almost all cultures had viabilities above 89% at the end of the culture. In contrary, cultures from the CHO-S cell line decreased more drastically below a viability of 82%. Interestingly, cells of the CHO-K1 cell lines experiencing a continuously produced media lead to higher number of total cells produced in comparison to the batch set-ups while that could not be observed for the CHO-S set ups. Also, the Glc concentration profiles were comparable for both cell lines irrespective of the production mode of the basal medium were also comparable. Also, the final titer in the end showed no differences between the batch wise and continuously reconstituted media (FIG. 5).

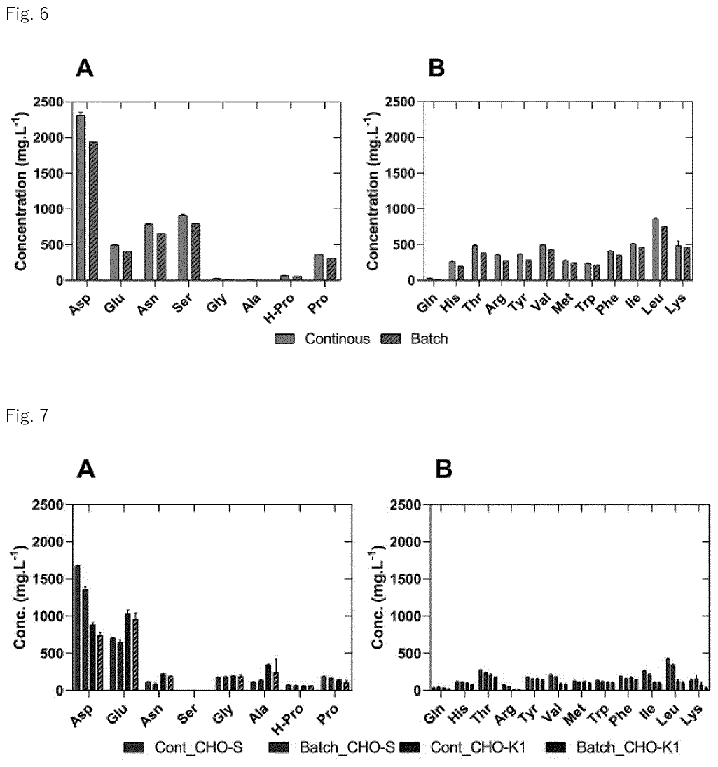

In addition, also the amino-acid profiles after reconstitution and at the end of the fed-batch experiments were analyzed (FIG. 6). The amino acid profiles between the different reconstitution modes indicates an almost comparable AA profile in respect to non-essential (NEAA) and essential (EAA) amino acids. As can be seen from FIG. 7 (A,B) also the amino acid profiles at the last of the cultivations show a relatively comparable amino acid profile within EAA and NEAA.

### Example 4—Continuous, On-Demand and Long-Term Reconstitution of a Chemically Defined Medium Directly from Solids for Fermentation Processes

Example 4 demonstrates the continuous on-demand reconstitution of chemically defined media directly from solids over a duration of 12 hours.

The feeding device comprised a screw conveyor, feeding hopper and a control unit capable of generating in-situ gradients. This continuous on-demand reconstitution of CDM can substantially shrink auxiliary buffer and media tanks needed for continuous upstream production like perfusion systems. Undoubtedly, CDM differs significantly in its powder characteristics and flow behaviour from buffer species commonly used in the biopharmaceutical industry simply due to their more complex formulations and manufacturing. For that reason, the device was adapted for the differences in powder flow behaviour by redesigning the geometrical shape, power translation and screw design.

The core unit of the developed system is a 3D printed powder feeder which continuously feeds dry powdered media in a continuous stirred tank reactor having an in and outflow (FIG. 1).

For the long-term reconstitution, a simple control unit loop was built which ensured a constant working volume (45 mL) in the mixing vessel and integrated the device into a confinement experiencing slight over-pressure with dry process air to prevent moisture accumulation. The device feeds solid particles into a mixing vessel which is consequently mixed in a tubular reactor. At the end of the tubular reactor, the now liquid medium is sterile filtered and either transported into a bioreactor or a hold tank. A peristaltic pump connected to an Arduino resupplied the stirring vessel with fresh RO-water and one dual piston pump of the ÄKTA system set at 6.9 mL min⁻¹ transported the medium through the tubular reactor as illustrated in FIG. 1. Dry medium powder was dosed at 0.171 g min⁻¹. The flow path of the ÄKTA system was set to by-pass for on-line monitoring of pH, UV and conductivity.

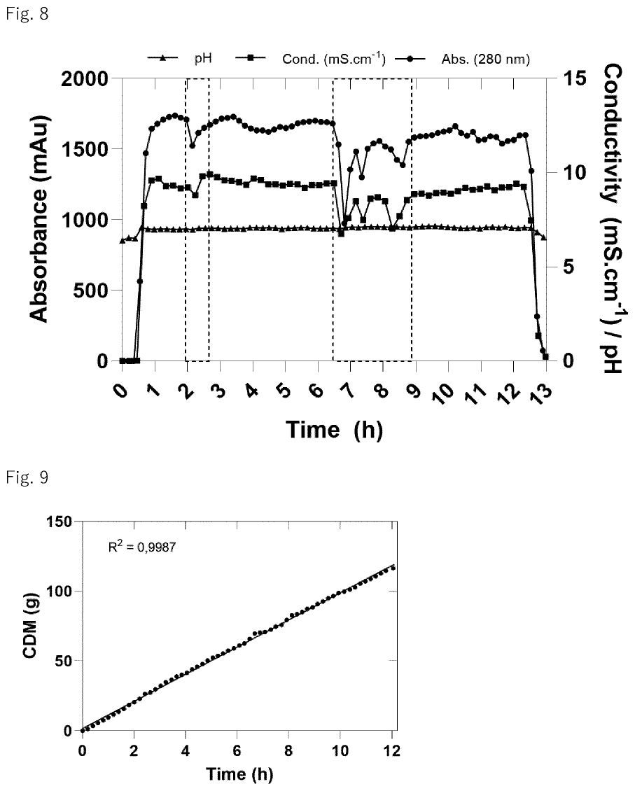

After a ramp up phase of 50 mins the continuous on-demand reconstitution entered stable conditions illustrated by the signal measured for the UV, conductivity and pH (FIG. 8). After two and six hours (FIG. 8, black dashed box) arching in the hopper and moisture accumulation occurred which could be resolved by manual removal of the arch and increasing the flow of the process air entering the confinement and thus restored the reconstitution to stable conditions. Thereby, the continuous reconstitution of chemically defined media on-demand for a duration of 12 h was reached.

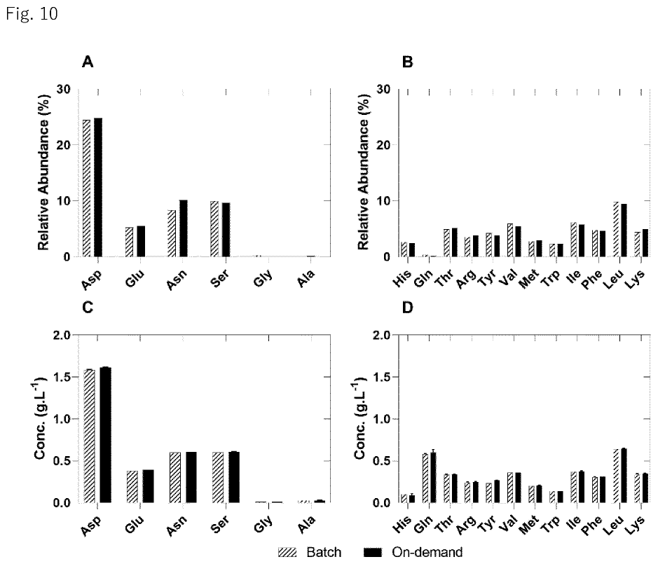

This and probably slight fluctuations in the volume of the CSTR with different dissolution kinetics of the individual components are most likely the reason for the fluctuations observed in the stable parts for the conductivity (±3.27%) and UV signal (±2.97%). Analysis of the feeding accuracy and precision as a function of weight over the duration of 12 h showed that the device, besides the two deviations, was within the expected accuracy for such small feeding rates (+/−5%) with a slightly lower but accurate feeding rate of 0.162 g min⁻¹ in contrast to the anticipated feeding rate of 0.171 g min⁻¹ (−5%). The robustness of the device is further demonstrated over the duration of the feeding of powder by tracking the fed amount of CDM by weight (FIG. 9). It is suggested to additionally implement on-line monitoring of weight and a control loop for the feeding of CDM as well as a control loop for the feeding of liquid into the system. Measurement of the osmolality for the on-demand collected medium during the stable part of the reconstitution resulted in in 276 mOsm Kg⁻¹ and 280 mOsm Kg⁻¹. Whereas, analysis of the conventional reconstituted medium resulted in a difference of 7% resulting in 296 mOsm Kg⁻¹ which is undoubtedly caused by the slightly lower feeding rate detected for the continuous reconstitution. Thus, to ensure same starting conditions, (+7%) the batchwise reconstituted media were diluted to the same osmolality using RO-water. To highlight any inconsistencies that might arise due to inhomogeneous dissolution of the CDM in comparison to the batch-wise reconstitution (e.g. non-uniform amino-acid dissolution leaving one or more components as solids), the amino acid composition after the reconstitution and at the day of inoculation of the continuously on-demand reconstituted media was analyzed (FIG. 10; A, B). As illustrated in FIG. 10 the relative abundance (A, B) of the individual amino acids and concentrations at the day of inoculation (C, D) after the continuous on-demand show a comparable profile to a conventional reconstituted media.

### Example 5—Bioreactor Experiments

A cell culture experiment was performed in a larger scale in controlled bioreactor conditions to study potential differences between the long-term continuous on-demand and batchwise reconstituted medium. For the experiment, the CHO-K1 cell line was used in controlled conditions using DASGIP bioreactors (n=2). For the bioreactor experiments, a DASGIP® Parallel bioreactor system was used (Eppendorf, Hamburg, Germany). The dissolved oxygen (DO) level was set at 50% and pH was controlled at 7.0 by gassing in of CO₂ using the DASGIP® modules and addition of sodium bicarbonate. Working volume was set at 0.7 L, respectively. Bioreactor experiments were carried out in biological duplicates. The media was equilibrated at process conditions for 6 h at 36.5° C. and stirrer speed was set at 150 rpm. For all fed batch experiments, cells were inoculated at a seeding cell density of 6.5×10⁵ cells mL⁻¹ with a 1:10 dilution of the spent media in fresh media. For the bioreactor experiments glucose levels were maintained above 4 g*L⁻¹ by daily bolus addition of a concentrated glucose stock solution (200 g*L⁻¹), if glucose concentration was <2g*L⁻¹. Notably, to prevent masking effects, no concentrated feed medium was introduced. A 1% solution of Antifoam C emulsion was added on demand.

### Process Analytics

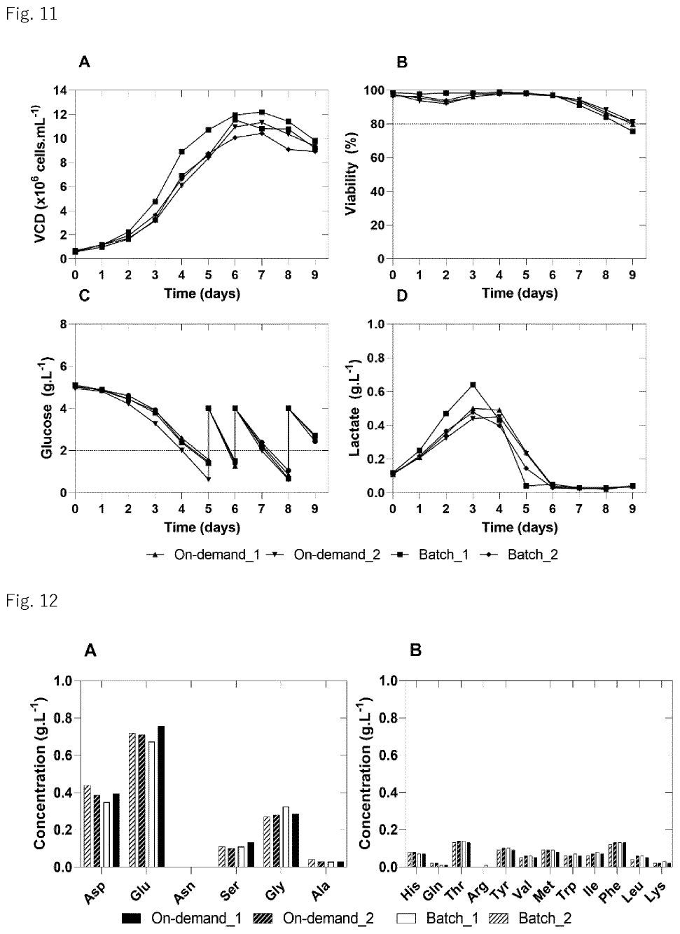

Likewise, to the spin-tube experiments, both approaches of medium reconstitution led to the same performance (FIG. 11). The pooled medium collection (On-demand_1, On-demand_2) of continuously on-demand reconstituted medium led to a maximum viable cell density (MVCD) of 11.53 and 11.32×10⁶ cells.mL⁻¹ and the cultures grown in batchwise reconstituted medium reached a MVCD of 12.19 and 10.43×10⁶ cells·mL⁻¹, respectively (FIG. 11).

Almost all cultures had viabilities around 80% at the day of harvest. Notably, the culture Batch_1 led to a slightly lower viability. Analysis of the final titer at the day of the harvest also led to a comparable performance of 0.193, 0.239, 0.235, 0.228 g L⁻¹ for On-demand_1, On-demand_2, Batch_1 and Batch_2. Also, analysis of the essential and non-essential amino acids at the day of harvest led to a comparable profile (FIG. 12).

### Product Quality

The antibodies were captured using preparative Protein A chromatography. HPLC Protein A affinity chromatography was used to determine the antibody concentration. To estimate product purity, size exclusion chromatography was performed. The antibody purity was calculated as the ratio of the monomer peak area (retention time 21.2 min) to the sum of all peak areas, based on the 280 nm signal. Intact protein analysis was performed using Sheathless CE-MS. Therefore, intact antibody samples were analyzed using a Sciex CESI 8000 instrument coupled via a XYZ stage to an Impact qTOF-MS (Bruker Daltonics, Bremen, Germany).

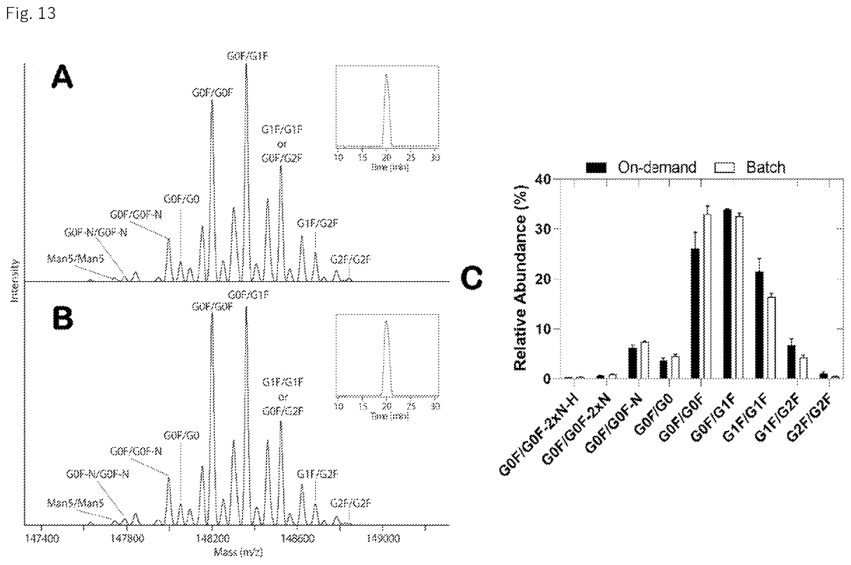

The product quality was analyzed and the post translational modifications (PTMs) of antibodies obtained with batch and on-demand reconstituted media were compared using sheathless CE-MS. FIG. 13—shows the results obtained after CE-MS analysis of the mAb samples. FIG. 13 shows deconvoluted mass spectra of (FIG. 13A) continuous on-demand reconstituted and (FIG. 13 B) batchwise reconstituted samples derived from controlled conditions after sheathless CE-MS separation. The inset shows the base peak electropherograms of both samples. FIG. 13 C shows the relative intensity of the glycoforms of mAbs produced with continuous on-demand reconstituted medium or batchwise reconstituted medium. No additional fragments were observed in the base peak electropherograms (FIG. 13). Furthermore, no differences in common modifications such as the presence of lysine variants or methionine oxidation could be observed. The glycosylation pattern between continuous on-demand and batchwise reconstitution showed only slight differences that are within expected batch to batch variations. Importantly, no significant differences in the levels of afucosylation between both conditions indicating comparable afucosylation (FIG. 13) were observed. Regarding other PTMs no differences were observed between the two evaluated conditions.

### Example 6—Continuous Reconstitution of a Chemically Defined Medium for Yeast Fermentations

Another example is the continuous reconstitution of medium for yeast fermentations for the production of citric acid using a chemostat process. Thereby, a chemically defined fermentation medium comprising NH₄Cl, glucose, NH₄Cl, KH₂PO₄, MgSO₄, MnSO₄, FeSO₄, CuSO₄, ZnSO₄, CoSO₄, H₃BO₃, CaCl, NaCl, citric acid, Na₂MoO₄ thiamine-HCl, biotin, pyridoxine-HCl, Ca-D-pantothenate and nicotinic acid was reconstituted and filtered through a 0.2 μm filter. Consequently, the medium was introduced into a chemostat cultivation. The reconstitution of this medium directly from solid can be realized by the use of the invention mentioned in this patent, thereby drastically reducing the necessary hold tanks for the media and expanding the possible control options during chemostat production.

### Example 7—Precision, Accuracy and Stability of Continuous Reconstitution of Buffers

Another example for the continuous reconstitution of process materials from solids is the use as buffer preparation units from solids without the need for large buffer tanks for the storage of said buffers. The reconstitution of five different buffering agents at various motor speeds and the reconstitution of NaCl at three different motor speeds is described in example 7.

### Experimental Setup

The feeding device was 3D printed and comprised a screw conveyor driven by stepper motors (Stepperonline, Nanjing, China). The device was controlled by a minicomputer Raspberry Pi 3 (Raspberry PI Foundation, Cambridge, United Kingdom) programmed using Python (Python Software Foundation, Wilmington, United States). For stability, precision and accuracy experiments the design of the hopper was optimized in regard to geometric shape. The solid compound was put into the storage tank of the feeding device and calibration experiments were conducted for sodium chloride (NaCl), tris(hydroxymethyl)aminomethane (Tris), sodium citrate monohydrate, polyethylen glycol 6000 (PEG 6000) and sodium acetate (NaAc). The calibration experiments were performed using an Entris® Precision balance (Sartorius, Gottingen, Germany) connected to the Raspberry Pi 3. The data was collected on-line using the Simple Data Logger software (Smartlux SARL, Born, Luxembourg).

### Precision, Accuracy and Stability

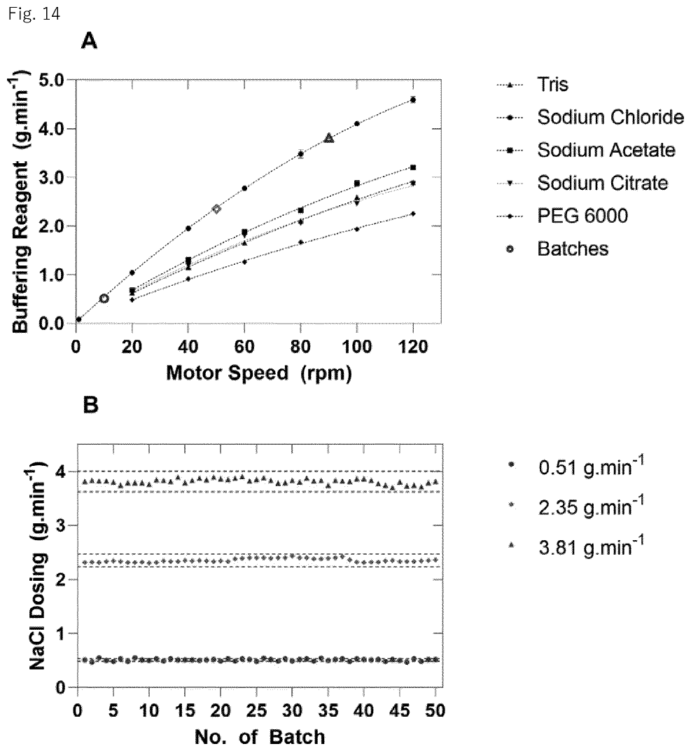

Precision and accuracy of solid feeding was evaluated by weight at different feeding speeds with sodium chloride, tris, sodium acetate, sodium citrate monohydrate, PEG 6000 and imidazole all in crystal form. The speed of the feeder was varied between 20-120 rpm. For sodium chloride, the range was increased from 1-120 rpm for the evaluation of long-term feeding. The actual feeding range in terms of g min⁻¹ differed between the tested chemicals. The range of the feeding rate was therefore dependent on the kind of solid, presumably due to different particle size, particle roughness and other physical properties, and the speed of the feeder needs to be adjusted to the kind of crystals to achieve the same gravimetric feeding rate. FIG. 14-(A) shows that for all components except imidazole the standard deviation was below 5% for all tested feeder speeds. Furthermore, it was noticed that the feeding rate in grams per revolution was highly dependent on the hygroscopic characteristics and the resulting bridging formation in the hopper, limiting the amount of crystal that is picked up by the screw conveyer itself. This led to difficulties to perform calibration curves for imidazole and sodium citrate monohydrate. To circumvent this limitation, a closed loop control was implemented into the python script controlling the feeder to automatically readjust the screw conveyer speed during runtime by measuring the weight of the hopper and feeder system. For sodium citrate monohydrate, regular manual tapping of the hopper resolved the issue of bridging formation and accurate and stable feeding rates were achieved (FIG. 14-A). Here, significant differences of feeding rates were seen for the investigated buffer components. While for Imidazole, an additional control by weight is necessary, it was not necessary to include additional control systems for any other compound. Since the calibration experiments resulted in a comparable performance throughout the various buffering agents, the system on feeding accuracy and precision of repeated batches (g min⁻¹, n=50) were tested for three different motor speeds using sodium chloride. As illustrated in FIG. 14-(B) all batches conducted at various motor speeds were in a ±5% range of the initial target feeding rate. Finally, to prove long term stability, a 24 h feeding was performed at a very low feeding rate of 0.05 g min⁻¹ to evaluate the stability of the system using sodium chloride and showed the stability of solid feed flow. A linear regression was performed over the duration of the feeding and using a confidence band of 95%. The standard error was very small (<5%) and was comparable to the shorter feeding times performed prior over a duration of 24 h. Therefore, it can be concluded that the invention presented herein offers precise and accurate performance in short term and long term and is therefore suitable for the reconstitution of buffers, for gradient generation for individual chromatography runs as well as for continuous long-term operation.

### Example 8—In-Situ Preparation Method of a Linear Salt Gradient Elution for Chromatographic Applications Directly from Solid Buffer Components

Buffers for ion exchange chromatography require adjusting the salt content during operation which is currently done by mixing two buffers of differing concentration to create a constantly changing salt gradient. This complex assembly can be replaced by continuously generating the buffer solution by the addition of more or less salt using the system and method according to the invention described herein. The reconstitution of a buffer with varying salt concentration directly from solids avoids any hold tanks necessary for storing two kinds of buffers of differing salt concentration and can be used directly from a water source to generate the necessary gradient. Example 8 describes the chromatographic separation of the two substances lysozyme and cytochrome c by applying a linear salt gradient for the elution of the substances from an ion exchange resin.

### Experimental Setup

The feeding device was 3D printed and comprised a screw conveyor driven by stepper motors (Stepperonline, Nanjing, China). The device was controlled by a minicomputer Raspberry Pi 3 (Raspberry PI Foundation, Cambridge, United Kingdom) programmed using Python (Python Software Foundation, Wilmington, United States). The solids were fed into a miniaturized continuously stirred tank reactor (CSTR) with a magnetic stirrer and bottom outlet. This reactor was connected to a short tubular reactor filled with static mixers and further connected to the ÄKTA purification system. Absorbance of UV and conductivity was measured using the sensors of the ÄKTA system. Conductivity and osmolality were confirmed using an offline MC226 conductivity meter (Mettler Toledo, Columbus, United States) and an OsmoTech® single sample osmometer (Advanced Instruments, Norwood, United States). The pH of the buffer solutions for the linear gradient was adjusted prior and confirmed manually at the end of the run.

### Linear Gradient Elution

In a small-scale chromatography experiment, a binary protein mixture consisting of cytochrome c and lysozyme was separated on a 1 mL cation exchanger by a linear gradient. As a scale up, a 10 mL cation exchanger was used. The solid feeding device was mounted on top of a small dynamic mixer connected to an ÄKTA Pure chromatography workstation. After sample application, a gradient elution was performed by adding salt in crystal form continuously to a liquid to achieve a gradient length of 10 CV. More specifically, the salt gradient elution experiments were carried out using Eshmuno CP-FT resin (Merck KGaA, Darmstadt, Germany). The small-scale experiments were performed using a prepacked Minichrom Eshmuno CP-FT resin with a volume of 1 mL. For the scale up, a Tricorn™ 10 housing (Cytiva, Uppsala, Sweden) was used with a final column volume of 10 mL Eshmuno CP-FT resin. Equilibration-, wash and elution buffer were 50 mM phosphate buffer pH 6.9 and 50 mM phosphate buffer supplemented with 500 mM sodium chloride for the elution buffer. Before loading, the column was equilibrated with 5 CV. Loading of the column was done using pulse injections with a loop volume of 100 μL. The feed concentration was 5 mg mL⁻¹ of lysozyme and cytochrome c dissolved in the equilibration buffer supplemented with 50 mM sodium chloride, respectively. All buffers were prepared either batch wise or by in-line conditioning directly from solids by the presented solid buffer preparation device which were consequently compared based on osmolality, conductivity and final pH. Absorbance of the elution fraction was measured at 280 nm for lysozyme and 405 nm for cytochrome C. For the in-line preparation directly from solid, sodium chloride was fed directly into a beaker with a working volume of 100 mL of phosphate buffer containing no additional salt. Once the in-line preparation was initiated, linearity was achieved by setting the feeding rate accordingly to the duration of the gradient.

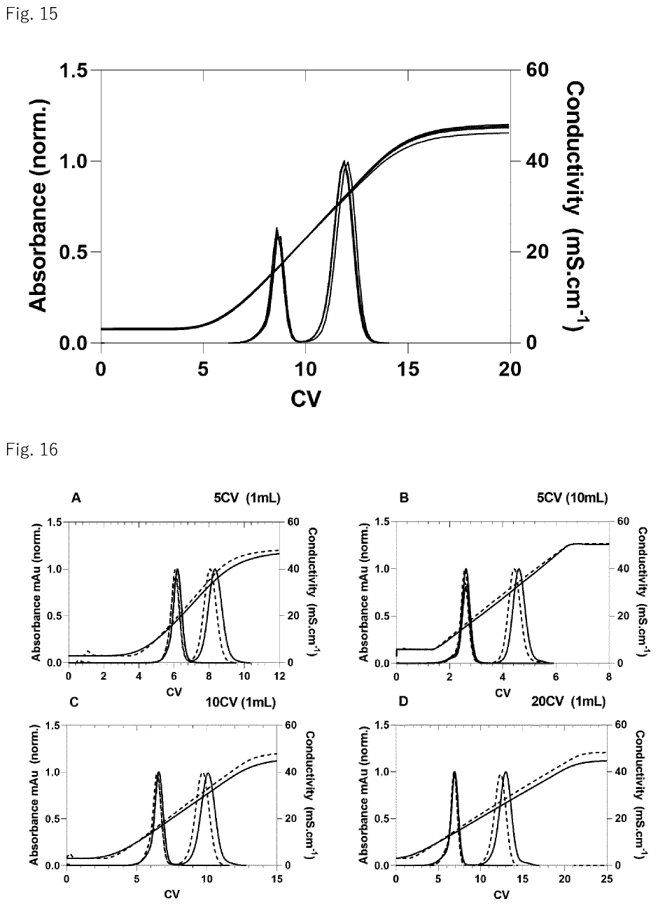

The capability of the device to perform stable and linear gradients was further evaluated and therefore, the gradient was repeated five consecutive times. The stability was evaluated based on the elution peaks of lysozyme and cytochrome c, conductivity as well as final osmolality (FIG. 15). The formation of the gradient by the presented method is highly reproducible. This can be shown by the average and standard deviation of the conductivities at peak maximum. For elution of cytochrome c, the average conductivity at peak maximum measured at 405 nm was 16.3±0.29 mS cm⁻¹; measured at 280 nm 16.4±0.35 mS cm⁻¹. This yields in a coefficient of variation of 1.7 and 1.8% respectively. For lysozyme a coefficient of variation of 1.3% (Elution conductivity at peak maximum is 31.9±0.41 mS cm⁻¹) was obtained. Also, the osmolality at the end of the gradient was measured and observed a coefficient of variation (947±18.2 mOsm kg⁻¹) of 1.9%.

For comparison, also a conventional run using two preprepared buffers was performed, and the gradient generation by pumps and in-line mixer which resulted in a final osmolality of 957±1.2 mOsm kg⁻¹. Then the device to perform gradients with different lengths (5, 10 and 20 CVs) was tested in 1 mL and 10 mL columns. The feeding rate was adjusted to the length of the gradient and the column volume. The salt gradients generated by the two systems pumps of the ÄKTA and the build-in mixer differed slightly at the beginning of the gradient (FIG. 16). The linearity of the gradient produced by the in-situ mixing from solid buffer components is comparable to the conventional buffer preparation (FIG. 16).

### Example 9—In-Situ Preparation Method of a Step Gradient Elution for Chromatographic Applications Directly from Solid Buffer Components

Example 9 describes a methodology for the generation of a step gradient for the chromatographic purification by the herein described invention. More specifically, a His-tagged protein was purified using a metal affinity chromatography resin, wherein the protein was eluted from the column by a buffer comprising imidazole. Imidazole is a very common buffer in metal chelate chromatography and therefore such a buffer was selected as model to demonstrate our in-situ gradient formation system. Thereby, a step gradient elution was developed directly from solid buffer components.

### Experimental Setup

The feeding device was 3D printed and comprised a screw conveyor driven by stepper motors (Stepperonline, Nanjing, China). The device was controlled by a minicomputer Raspberry Pi 3 (Raspberry PI Foundation, Cambridge, United Kingdom) programmed using Python (Python Software Foundation, Wilmington, United States). The solids were fed into a miniaturized continuously stirred tank reactor (CSTR) with a magnetic stirrer and bottom outlet. This reactor was connected to a short tubular reactor filled with static mixers and further connected to the ÄKTA purification system. Absorbance of UV and conductivity was measured using the sensors of the ÄKTA system. Conductivity and osmolality were confirmed using an offline MC226 conductivity meter (Mettler Toledo, Columbus, United States) and an OsmoTech® single sample osmometer (Advanced Instruments, Norwood, United States). The pH of the buffer solutions for step gradients was adjusted prior and confirmed manually at the end of the run.

### Step Gradient Elution

The feeding device was mounted on a vessel filled with base buffer which was used for equilibration and sample application to the immobilized metal affinity chromatography column. For step gradient elution experiments, an immobilized metal affinity resin Ni Sepharose 6 Fast Flow (Cytiva, Uppsala, Sweden) resin was used. Step gradient experiments were performed in a Tricorn™ 10 housing (Cytiva, Uppsala, Sweden) with a column volume of 2.1 mL. Equilibration and wash buffer were 50 mM phosphate buffer pH 8.0 supplemented with 10 mM imidazole and 300 mM sodium chloride. The elution buffer was supplemented with imidazole to reach a concentration of 500 mM. Before loading, the column was equilibrated with 5 CV and washed after the loading step with 2 CV. Loading of the column was done using pulse injections with a loop volume of 100 μL. The feed concentration of His-tagged green fluorescent protein (GFP) in the equilibration buffer was 2.2 mg mL⁻¹. All buffers were prepared either batch wise or in-line by the presented solid buffer preparation device which were consequently compared based on osmolality, conductivity and final pH. Absorbance of the elution fraction was measured at 488 nm for GFP and 240 nm for blank gradient experiments. For the in-line preparation of equilibration buffer, imidazole was fed based on a scale into a beaker to reach an equilibration buffer concentration of 10 mM imidazole. After loading of the sample onto the column additional imidazole was fed into the beaker to reach a target concentration of 500 mM imidazole. After the equilibration, a His-Tag GFP solution was loaded on the column by pulse injection. To generate the step gradient, imidazole was fed with maximum speed (200 rpm) into the buffer reservoir to reach 500 mM imidazole as fast as possible. The feeding was done by weight to ensure a steep gradient for elution due to the hygroscopicity of imidazole. The ÄKTA was on pause over the duration of the feeding (10 minutes).

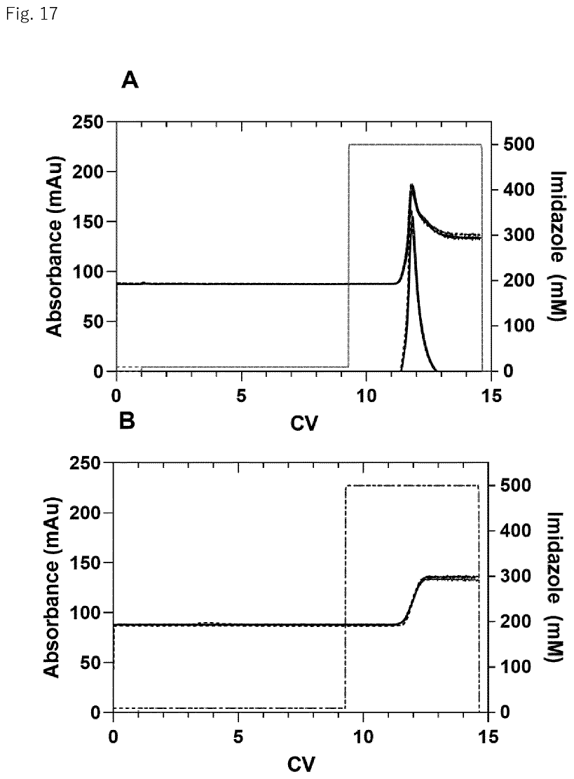

The step gradient performed by the ÄKTA using the in-line mixer and the device for in situ preparation from solid buffer components approach resulted in almost identical chromatograms (FIG. 17-A). These findings are further supported by the final osmolality of the equilibration (674.0±3.28 mOsm kg⁻¹ device for in situ formation from solid buffer components vs. 670.50±14.41 mOsm kg⁻¹ ÄKTA) and elution buffer (1160±19.55 mOsm kg⁻¹ device for in situ formation from solid buffer components vs. 1190.52±1.70 mOsm kg⁻¹), respectively. Since imidazole can be readily measured by UV absorbance, comparisons without protein solution were performed (FIG. 17-B).

1. A system for on-demand reconstituting solid process material, comprising
a. a feeding device for continuously feeding said solid process material, wherein the feeding rate can be directly regulated during operation of the feeding device by adjusting the feeding device and wherein said feeding device is driven by a motor;
b. a mixing vessel;
c. optionally a hold tank;
d. optionally one or more mixing reactors; and
e. optionally a sterile filter unit,
wherein the system is configured to operate continuously.

2. (canceled)

3. The system of claim 1, wherein said system is connected to a reactor.

4. The system of claim 1, wherein said solid process material is a cell culture medium, a buffer, a nutrient, an additive, a substrate, a salt, a polymer, a chemical, a bulk material, or any combination thereof.

5. The system of claim 1, wherein said solid process material is an organic and/or inorganic material, and wherein said process material is in the form of a powder, a slurry, a crystal, an organic polymer, an inorganic polymer, or a granulate.

6. The system of claim 1, wherein said feeding device is selected from the group consisting of a screw conveyor, extruder, apron conveyor, pneumatic conveyor, roller conveyor, belt conveyor, pelletizer, compounder, gravimetric feeder, acoustic and ultrasonic vibration conveyor, rotary conveyor, electromagnetic conveyor, and vertical conveyor.

7. The system of claim 1, wherein a feeding hopper is connected to said feeding device.

8. (canceled)

9. The system of claim 1, wherein the feeding device is comprised in a confinement.

10. The system of claim 9, wherein the confinement is flushed by gas either with or without an overpressure.

11. The system of claim 1, further comprising one or more tubular reactors as mixing reactors.

12. The system of claim 1, further comprising one or more integrated sensors for assessing process parameters.

13. The system of claim 12, wherein said one or more sensors are selected from the group consisting of a temperature sensor, a pH sensor, a flow rate sensor, a concentration sensor, a fluorescence sensor, an infrared light sensor, a sensor for inelastic scattering of monochromatic light, a conductivity sensor, a redox potential sensor, a pressure sensor, an air moisture sensor, and a biomass sensor.

14. The system of claim 1, wherein said reactor is a fermentation bioreactor operated in batch mode, fed-batch mode or continuous mode.

15. (canceled)

16. A method for reconstituting a process material on-demand in a continuous mode, comprising:
a. providing the system of claim 1;
b. adding a solid process material in a continuous mode to the mixing vessel;
c. adding liquid in a continuous mode to the mixing vessel;
d. allowing said solid process material to mix with said liquid in the mixing vessel to provide reconstituted process material; and
e. transferring the reconstituted process material in a continuous mode to the reactor.

17. The method according to claim 16, wherein said liquid is water, dissolved buffer in a solvent, dissolved chemically defined medium in a solvent, or recycled process stream.

18. The method of claim 16, wherein said liquid is provided from a hold tank or from a reactor.

19. The method of claim 16, wherein the solid process material is a cell culture medium, a buffer, a nutrient, an additive, a substrate, a salt, a polymer, a chemical and/or a bulk material, or any combination thereof, and wherein said solid process material is in the form of a powder, a slurry, a crystal, an organic polymer, an inorganic polymer or a granulate.

20. The method of claim 16, wherein the continuous operation mode is performed for at least 12 hours.

21. The method of claim 16, wherein batch-wise production of the process media is avoided.

22. The method of claim 16, wherein said reactor is a bioreactor comprising mammalian cells, bacterial cells, insect cells, fungal cells, algae or yeast for producing a product.

23. The method of claim 22, wherein the product is a peptide, a protein, an oligonucleotide, a polynucleotide, a protein conjugate, a virus, a virus like particle, an exosome, a microorganism, a cell, or a tissue.
